(19) United States
(12) Patent Application Publication
(10) Pub. No.: US 2025/0040828 A1
(43) Pub. Date: Feb. 6, 2025

(54) CARDIAC MAPPING SYSTEMS AND METHODS WITH INTEGRATED NAVIGATION MODES

(71) Applicant: St. Jude Medical, Cardiology Division, Inc., St. Paul, MN (US)

(72) Inventors: Hai Huang, Woodbury, MN (US); Ashish Bhargava, Woodbury, MN (US); Kristi M. Tanouye, Minneapolis, MN (US); Shuaidong Zhao, Woodbury, MN (US)

(21) Appl. No.: 18/792,149

(22) Filed: Aug. 1, 2024

Related U.S. Application Data
(60) Provisional application No. 63/530,758, filed on Aug. 4, 2023.

(51) Int. Cl. A61B 5/06

(52) U.S. Cl. CPC A61B 5/063; A61B 5/062

(57) ABSTRACT

A method to collect and present cardiac medical information to a physician using a catheter includes receiving impedance-based position data from an electrode. A first navigation mode for visualizing the catheter is generated using the impedance-based position data. Magnetic-based position data is received from a magnetic sensor. A second navigation mode for visualizing the catheter is generated using the magnetic-based position data. A determination is made regarding whether the catheter is within a magnetic motion box generated by a magnetic field generator. One or more of the magnetic-based position data and the impedance-based position data is used to determine whether the catheter is within the magnetic motion box. The method includes outputting the first navigation mode to a display when the catheter is outside the magnetic motion box and outputting the second navigation mode to the display when the catheter is inside of the magnetic motion box.

### CROSS-REFERENCE TO RELATED APPLICATIONS

This application claims the benefit of and priority to U.S. Provisional Application No. 63/530,758 titled “CARDIAC MAPPING SYSTEMS AND METHODS WITH INTEGRATED NAVIGATION MODES,” filed Aug. 4, 2023, the contents of which are incorporated by reference herein.

### BACKGROUND

The present disclosure relates generally to medical positioning systems devices, systems, and methods for localization for selecting between impedance-based navigation and magnetic-based navigation to visualize a catheter within the body.

Medical positioning systems (MPS), such as impedance-based positioning systems and magnetic-based positioning systems, are used to determine the position and/or orientation of a medical device within a patient's body. Other methods of monitoring the position/orientation of a medical device within the body include utilizing fluoroscopic radiation (e.g., x-ray radiation). However, neither impedance-based navigation nor magnetic-based navigation require exposure to x-ray radiation and therefore are preferable in some applications.

A typical electro-anatomical 3D navigation system includes a two-dimensional image acquisition system (e.g., ultrasound or low-fluoroscopic imagining system), a medical positioning system (MPS) (i.e., a location and orientation detection system), and an image processing system. The imaging system reconstructs a three-dimensional image from all the recorded two-dimensional images. The system displays a sequence of these three-dimensional images, synchronized with a real-time reading of the organ timing signal, thereby providing a real-time visualization of the inspected organ. The physician can insert a minimally invasive surgical tool (e.g., a sensor enabled catheter) into the body of the patient. The system detects the location and orientation of the MPS sensor mounted on the surgical tool and super-imposes a representation thereof, on the currently displayed three-dimensional image. Thus, the electro-anatomical 3D navigation system enables a physician to visualize the surgical tool relative to the inspected organ to aid the physician in administering a medical treatment.

### SUMMARY

According to a first aspect of the invention, a method to collect and present cardiac medical information to a physician using a medical device includes receiving impedance-based position data from an electrode. A first navigation mode (or “impedance-based navigation mode”) for visualizing the medical device is generated using the impedance-based position data. Magnetic-based position data is received from a magnetic sensor. A second navigation mode (or “magnetic-based navigation mode”) for visualizing the medical device is generated using the magnetic-based position data. A determination is made regarding whether the medical device is within a magnetic motion box generated by a magnetic field generator. One or more of the magnetic-based position data and the impedance-based position data is used to determine whether the medical device is within the magnetic motion box. The method includes outputting the first navigation mode to a display when the medical device is outside the magnetic motion box and outputting the second navigation mode to the display when the medical device is inside of the magnetic motion box.

According to a second aspect of the invention, a medical imaging and navigation system includes a medical device, a pair of surface patch electrodes, a magnetic field generating assembly, a display, and a processor. The medical device includes a device electrode and a magnetic sensor. The pair of surface patch electrodes are positionable on a patient. The pair of surface patch electrodes are configured to generate an electric field within a patient body. A first electrical signal is measured on the device electrode due to the generated electric field. The magnetic field generating assembly is positioned adjacent the patient. The magnetic field generating assembly generates a magnetic field within a magnetic motion box. A second electrical signal is measured on the magnetic sensor due to the generated magnetic field. The processor receives the first electrical signal from the device electrode and determines a first position point of the device electrode based on the first electrical signal. The processor stores the first position point in a first dataset. The processor receives the second electrical signal from magnetic sensor and determines a second position point of the magnetic sensor based on the second electrical signal. The processor stores the second position point in a second dataset. The processor utilizes the first dataset to construct an impedance-based map study and utilizes the second dataset to construct a magnetic-based map study. The processor selectively outputs either the impedance-based map study or the magnetic-based map study to the display based upon a location of the device within the patient body. The processor is configured to transition from the impedance-based map study to the magnetic-based map study within a single study.

These and other examples and features of the present devices, systems, and methods will be set forth, at least in part, in the following Detailed Description. This Overview is intended to provide non-limiting examples of the present subject matter-it is not intended to provide an exclusive or exhaustive explanation. The Detailed Description below is included to provide further information about the present devices, systems, and methods.

### BRIEF DESCRIPTION OF DRAWINGS

This written disclosure describes illustrative embodiments that are non-limiting and non-exhaustive. Reference is made to illustrative embodiments that are depicted in the figures, in which:

FIG. 1 is a systematic and diagrammatic view of an exemplary medical system for performing one or more diagnostic or therapeutic procedures, wherein the system includes a medical positioning system, according to some embodiments.

FIG. 2A is a schematic view of a sensor enabled catheter inserted within a patient's body, according to some embodiments.

FIG. 2B is a magnified view of a sensor enabled catheter, according to some embodiments.

FIG. 3 is a flow chart of a method of selecting the navigation mode to display to a physician, according to some embodiments.

FIG. 4 is a method of determining whether a catheter is located within a patient's body, according to some embodiments.

FIG. 5A is a flow chart of a method of determining whether a catheter is located within a magnetic motion box, according to some embodiments.

FIG. 5B is a flow chart of a method of determining whether a catheter is located within a magnetic motion box, according to some embodiments.

FIG. 5C is a flow chart of a method of determining whether a catheter is located within a magnetic motion box, according to some embodiments.

### DETAILED DESCRIPTION

According to some embodiments, the present disclosure describes systems and methods for providing medical positioning. In particular, a medical positioning system operates in an impedance-based navigation mode (also referred to as a “first navigation mode”) and a magnetic-based navigation mode (also referred to as a “second navigation mode”). The medical positioning system selectively operates in either the impedance-based navigation mode or the magnetic-based navigation mode based on the determined position of the catheter.

Impedance-based navigation (or “localization”) allows immediate visualization of the position of a device having one or more electrodes inserted within a patient's body. The impedance field (or electric field) may be generated within a patient's body by applying a pair (or multiple pairs) of surface patch electrodes to a patient body and driving an electrical current through the pair of surface patch electrodes. A catheter including one or more electrodes senses the impedance field and utilizes the measured response to the impedance field to determine the position of the catheter within the patient's body. In some embodiments, a benefit of impedance-based navigation is that the position of the electrode is determined immediately upon entrance into the patient's body and can thus be utilized to visualize the catheter anywhere within a patient. Immediate visualization is beneficial, as it allows the physician to visualize the catheter as the physician urges the catheter from the point of insertion toward the target area. However, there are drawbacks to impedance-based navigation. For example, the electric field (or impedance field) may fluctuate from patient-to-patient and/or may fluctuate within a single procedure as anatomic conditions change. Further, the electric field may be non-linear, as bones, fats, and triglycerides in the body have a high impedances whereas bodily fluids and cell membranes have low impedances.

Magnetic-based navigation may also be utilized to determine the location and/or orientation of a magnetic sensor within the patient's body and may provide improved accuracy over impedance-based navigation systems. However, magnetic-based navigation is typically only available within a specified area that corresponds with a stable magnetic field. This area is sometimes referred to as a magnetic motion box, wherein magnetic localization within the magnetic motion box is highly accurate and magnetic localization outside of the magnetic motion box is less accurate.

Magnetic-based navigation relies on a magnetic sensor located on the catheter sensing an external magnetic field. The magnetic field may be generated by a magnetic field generator assembly (e.g., located within the operating table). Typically, the magnetic field is optimized (e.g., stable, linear) within a particular 3D space relative to the magnetic field generator assembly. Positioning of the magnetic field generator assembly with respect to the target area (e.g., heart) associated with the patient allows for the magnetic-based navigation system to provide accurate location and/or orientation information within the defined 3D space or volume, referred to as the magnetic motion box. A catheter including a magnetic-enabled sensor may be placed within the magnetic field or motion box and the magnetic field may impart an electrical signal to the magnetic-enabled sensor. The electrical signal may be sent to a processor, and the processor may compute a position and orientation of the electrode based on the received electrical signal.

Magnetic-based navigation (or “localization”) provides accurate position and/or orientation sensing within the magnetic motion box but may provide less accurate position and/or orientation sensing when the magnetic sensor is outside of the magnetic motion box. In this way, in contrast with impedance-based navigation, magnetic-based navigation may not be available (or may be less accurate) upon the catheter initially entering the body but may provide improved accuracy when the catheter is within the magnetic motion box. Therefore, it would be beneficial to utilize the impedance-based navigation immediately upon catheter entry into the patient body, and transition to magnetic-based navigation upon catheter entry into the motion box.

This disclosure describes a system and method to integrate the impedance-based navigation mode with the magnetic-based navigation mode to allow automatic transitions and/or on-demand transitions (initiated by a user) between the impedance-based navigation mode and the magnetic-based navigation mode within a single study. Thus, the mapping/navigation system described herein is capable of immediate visualization upon the catheter entering a patient's body, and capable of stable, high-precision, magnetic-based navigation.

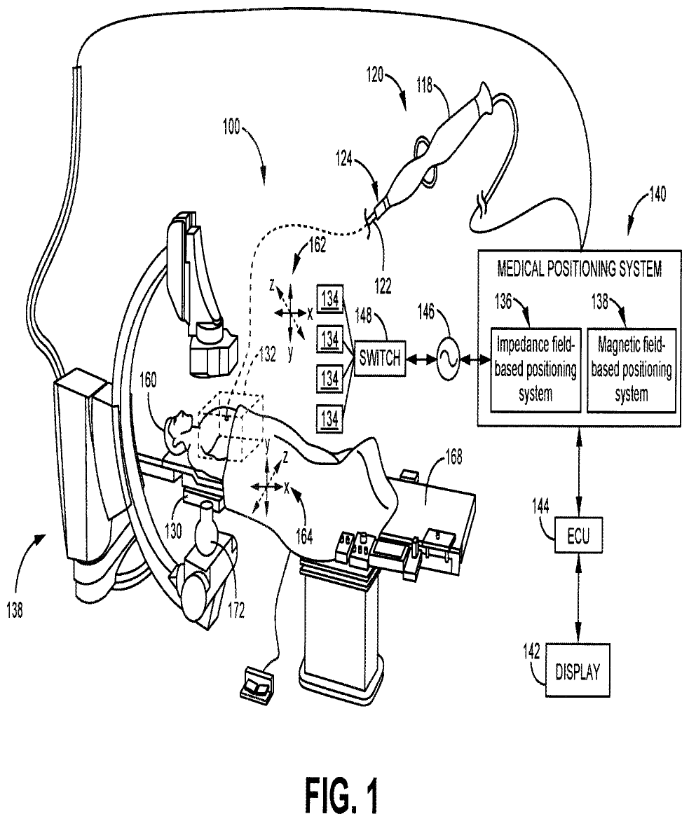

FIG. 1 illustrates a systematic and diagrammatic view of an exemplary medical system 100 for performing one or more diagnostic or therapeutic procedures, wherein the system includes a medical positioning system 140, according to some embodiments. The medical system 100 can include a medical device 120 and a medical positioning system 140. In some embodiments, the medical device 120 includes a handle 118, a shaft 122 having a proximal end 124 and a distal end 126. In some embodiments, the medical positioning system 140 includes an impedance field-based positioning system 136 and a magnetic field-based positioning system 138. In some embodiments, the impedance based positioning system includes a plurality of surface patch electrodes 134 are adhered to the skin of the patient 160, a switch 148 and alternating current (AC) source 146 connected through switch 148 to the plurality of surface patch electrodes 134. In some embodiments, the magnetic field based positioning system 138 includes a magnetic field generator 130 positioning adjacent to the patient and configured to generate a magnetic field in an area surrounding the patient (in particular, in an area designated the magnetic motion box 132). An electronic control unit (ECU) is connected to the medical positioning system 140 and is configured to receive impedance based positioning estimates and magnetic based positioning estimates associated with the one or more sensors located at the distal end 126 of the catheter 120. In some embodiments, the ECU 144 determines—based on one or more inputs including in some embodiments the impedance based position estimates and/or the magnetic field based position estimates—whether to operate in an impedance based mode in which the position of the catheter displayed via display 142 is based on the impedance based measurements or in a magnetic based mode in which the position of the catheter displayed via display 142 is based on the magnetic based measurements.

As shown in FIG. 1, the medical device 120 can include an elongated medical device such as, for example, a catheter or a sheath. For purposes of illustration and clarity, the description below will be limited to an embodiment wherein the medical device 120 comprises a catheter (e.g., catheter 120). It will be appreciated, however, that the present disclosure is not meant to be limited to such an embodiment, but rather in other example embodiments, the medical device may comprise other medical devices, such as, for example and without limitation, sheaths, guidewires and the like. In yet further embodiments, the medical device 120 may be any medical device wherein real-time location-based data may be advantageous to a procedure in which it is used.

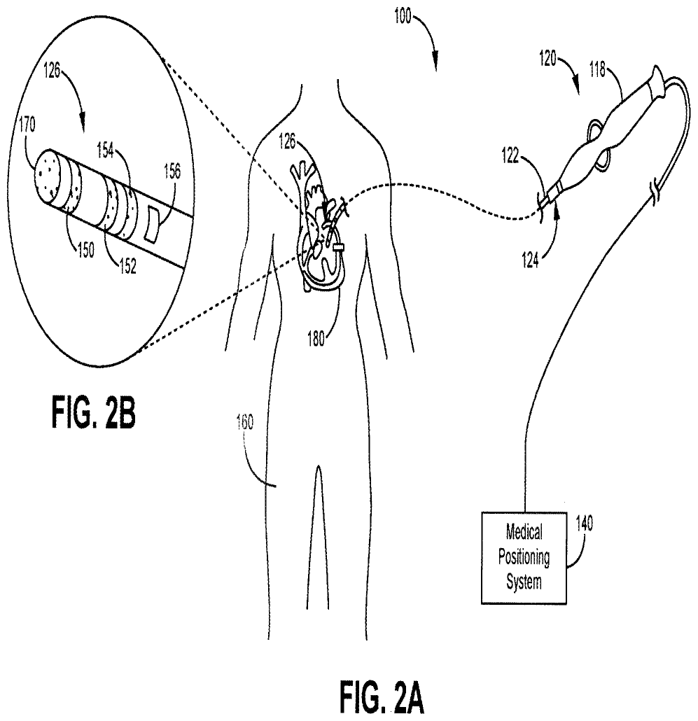

A distal end 126 (shown in FIG. 2A) of catheter 120 can be inserted into a patient's body 160, and more particularly, into the patient's heart 180 (also shown in FIG. 2A). The catheter 120 may include a handle 118, a shaft 122 having a proximal end 124 and a distal end 126, and one or more sensors (for example, sensors 150, 152, 154, 156, and 170 shown in FIG. 2B) mounted in or on the shaft 122 of the catheter 120. As used herein, “sensor 150” or “sensors 150” may refer to one or more sensors as appropriate and as generally depicted. The sensors 150 may include a catheter electrode and/or a magnetic sensor. In an exemplary embodiment, the sensors 150 are disposed at the distal end 126 of the shaft 122. Monitoring by the one or more sensors 150 are communicated via the shaft 122 to the handle 118 and are received by the medical positioning system 140. As described in more detail below, signals received from the one or more electrodes (e.g., electrodes 152, 154, 170) are utilized by the impedance based-positioning system to determine the position of the electrodes within the patient. Likewise, signals received from the magnetic sensor 156 are utilized by the magnetic based-positioning system to determine the position of the magnetic sensor 156 within the patient. In some embodiments, both the impedance based-positioning system 136 and the magnetic based-positioning system 138 generate corresponding position estimates. In some embodiments, ECU 144 receives both impedance based position estimates and magnetic based position estimates (and in some embodiments, additional signals measured by one or more sensors located on the catheter 120) and utilizes the received estimates to determine whether to operate in an impedance based mode in which the position of the catheter 120 is visualized to display 142 based on impedance based position estimates or in a magnetic based mode in which the position of the catheter 120 is visualized to display 142 based on magnetic based position estimates.

The catheter 120 may further include other conventional components such as, for example and without limitation, a temperature sensor, additional sensors or electrodes, ablation elements (e.g., ablation tip electrodes for delivering RF ablative energy, high intensity focused ultrasound ablation elements, etc.), and corresponding conductors or leads.

The shaft 122 can be an elongated, tubular, flexible member for movement within the body 160. The shaft 122 supports, for example and without limitation, sensors and/or electrodes mounted thereon, such as, for example, the sensors 150, associated conductors, and possibly additional electronics used for signal processing and conditioning. The shaft 122 may also permit transport, delivery, and/or removal of fluids (including irrigation fluids, cryogenic ablation fluids, and bodily fluids), medicines, and/or surgical tools or instruments. The shaft 122 may be made from conventional materials such as polyurethane, and define one or more lumens configured to house and/or transport electrical conductors, fluids, or surgical tools. The shaft 122 may be introduced into a blood vessel or other structure within the body 160 through a conventional introducer. The shaft 122 may then be steered or guided through the body 160 to a desired location, such as the heart 180, using means well known in the art.

The sensors 150 mounted in or on the shaft 122 of the catheter 120 may be provided for a variety of diagnostic and therapeutic purposes including, for example and without limitation, electrophysiological studies, pacing, cardiac mapping, and ablation. In an example embodiment, one or more of the sensors 150 are provided to perform a location or position sensing function. More particularly, and as described in greater detail below, one or more of the sensors 150 can be a positioning sensor that provides information relating to the location (e.g., position and orientation) of the catheter 120, and the distal end 126 of the shaft 122 thereof, in particular, at certain points in time. Accordingly, in such an embodiment, as the catheter 120 is moved along a surface of a structure of interest of the heart 180 and/or about the interior of the structure, the sensor(s) 150 can be used to collect location data points that correspond to the surface of, and/or other locations within, the structure of interest. These location data points can then be used for a number of purposes such as, for example and without limitation, the construction of surface models of the structure of interest. For purposes of clarity and illustration, the description below will be with respect to catheter 120 wherein a single sensor 150 comprises a positioning sensor. It will be appreciated, however, that in other example embodiments, which remain within the spirit and scope of the present disclosure, the catheter 120 may comprise more than one positioning sensor as well as other sensors or electrodes configured to perform other diagnostic and/or therapeutic functions, for example to find the six degrees of freedom of the catheter. As will be described in greater detail below, the sensor 150 can include a pair of leads extending from a sensing element thereof (e.g., a coil) that electrically couple the sensor 150 to other components of the system 100, such as, for example, the medical positioning system 140.

Some embodiments of the medical positioning system 140 will now be described. The medical positioning system 140 can be provided for determining a position and/or orientation of the sensor 150 of the catheter 120, and thus, the position and/or orientation of a distal portion of the catheter 120. In some embodiments, the medical positioning system 140 may comprise a magnetic field-based system such as, for example, the MediGuide™ system from MediGuide Ltd. (now owned by St. Jude Medical, Inc.), and as generally shown with reference to one or more of U.S. Pat. Nos. 6,233,476; 7,197,354; and 7,386,339, the entire disclosures of which are incorporated herein by reference.

In some embodiments, the medical positioning system 140 comprises, at least in part, a magnetic field generator 130 for generating a magnetic field for tracking of an object (e.g., a distal portion of catheter 120). The magnetic field generator 130 can generate a low-strength magnetic field(s) in and around the patient's chest cavity (e.g., an area of interest during a cardiac surgical procedure), which can be defined as a three-dimensional space designating a magnetic motion box 132 (also referred to as the “area of interest”). In such an embodiment, and as briefly described above, the catheter 120 includes a positioning sensor 150 comprising a magnetic sensor (e.g., a magnetic sensor 156 illustrated in FIG. 2B) that detects one or more characteristics of the low-strength magnetic field(s) applied by the magnetic field generator 130 when the sensor 150 is disposed within the magnetic motion box 132. The sensor 150, which in an exemplary embodiment comprises a magnetic coil, can be electrically/communicatively coupled with processing circuitry of a medical positioning system 140. The signals received by the processing circuitry corresponds to the sensed characteristics of the magnetic field(s) to which the magnetic coil is exposed. The processing circuitry, responsive to the detected signal, calculates a three-dimensional position and/or orientation for the sensor 150 and an input to the magnetic field generator 130. Thus, the medical positioning system 140 enables real-time tracking of each magnetic sensor 150 of the catheter 120 in three-dimensional space, and thereby, real-time tracking of the catheter 120.

The magnetic-field-based positioning system 138 in this exemplary embodiment employs magnetic fields to detect the position and orientation of the catheter 120 within the body 160. The system 138 may include the GMPS system made available by MediGuide, Ltd. and generally shown and described in, for example, U.S. Pat. No. 7,386,339 titled “Medical Imaging and Navigation System,” the entire disclosure of which is hereby incorporated by reference as though fully set forth herein. In such a system, a magnetic field generator 130 may be employed having three orthogonally arranged coils (not shown) to create a magnetic field within the body 160 and to control the strength, orientation, and frequency of the field. The magnetic field generator 130 may be located above or below the patient (e.g., under a patient table) or in another appropriate location. Magnetic fields are generated by the coils and current or voltage measurements for one or more position sensors 150 (e.g., the magnetic sensor 156) associated with the catheter 120 are obtained. The measured currents or voltages are diminishing functions of the distance of the sensors from the coils, thereby allowing determination of a position of the sensors within a second coordinate system 164 of system 138.

In some embodiments, the magnetic field generator 130 can be located underneath a patient examination table 168, between an x-ray source 172 and the patient examination table 168. For example, the magnetic field generator 130 can be coupled to the patient examination table 168. In some embodiments, as discussed herein, the magnetic field generator 130 can be a mobile device, which can be placed on a chest of the patient and used to generate the magnetic field for tracking of the object. In yet other embodiments, aspects of the present disclosure can be directed to a magnetic field generator 130 which includes one or more sensors on various sides of the magnetic motion box 132 of a patient 160. In such an embodiment, magnetic field distortions within (and external to) the area of interest may be cancelled by, for example, producing opposing magnetic fields on opposite sides of a distorting object, and thereby cancelling a magnetic field at the location of the distorting object. In yet other embodiments, magnetic field transmitters may be positioned at various locations around the magnetic motion box 132 to create varying magnetic field orientations that reduce an eddy from the distorting object.

In some embodiments, the medical positioning system 140 comprises, at least in part, one or more surface patch electrodes 134 for generating an electric field for tracking of an object (e.g., a distal portion of catheter 120). The surface patch electrodes 134 can generate an electric field(s)/impedance field in and around the patient's body 160. The surface patch electrodes 134 may be positioned on and/or around the patient's body 160 to generate an impedance-based coordinate system. The catheter 120 may include a positioning sensor 150 comprising an electrode or electrode pair (e.g., the electrodes 152, 154) that detect one or more characteristics of the electric field(s) applied by the surface patch electrodes 134 when the sensor 150 is disposed within the patient body 160. The sensor 150, which in an exemplary embodiment, comprises an electrode/electrode pair, can be electrically/communicatively coupled with processing circuitry of a medical positioning system 140. The signals received by the processing circuitry corresponds to the sensed characteristics of the electric field(s) to which the electrode is exposed. The processing circuitry, responsive to the detected signal, calculates a three-dimensional position and/or orientation for the sensor 150 and an input to the surface patch electrodes. Thus, the medical positioning system 140 enables real-time tracking of each electrode sensor 150 of the catheter 120 in three-dimensional space, and thereby, real-time tracking of the catheter 120.

The system 100 may include an electric-field-based positioning system 136, a magnetic-field-based positioning system 138, a display 142, and an electronic control unit (ECU) 144. Each of the exemplary system components is described further below.

The electric-field-based positioning system 136 (also referred to as the “impedance-based positioning system”) may be provided to determine the position and orientation of the catheter 120 and similar devices within the body 160. The system 136 may comprise, for example, the ENSITE NAVX system sold by St. Jude Medical, Inc. of St. Paul, Minn., and described in, for example, U.S. Pat. No. 7,263,397 titled “Method and Apparatus for Catheter Navigation and Location Mapping in the Heart,” the entire disclosure of which is hereby incorporated by reference as though fully set forth herein. The system 136 operates based upon the principle that when low amplitude electrical current signals are passed through the thorax, the body 160 acts as a voltage divider (or potentiometer or rheostat) such that the electrical potential measured at one or more electrodes 150 (e.g., the electrodes 152, 154) on the catheter 120 may be used to determine the position of the electrodes 150, and, therefore, of the catheter 120, relative to a pair of external patch electrodes 134 using Ohm's law and the relative location of a reference electrode (e.g., in the coronary sinus).

The electric-field-based positioning system 136 may further include multiple pairs of patch electrodes 134, which are provided to generate electrical signals used in determining the position of the catheter 120 within a first three-dimensional coordinate system 162. The electrodes 134 may also be used to generate impedance-based position data regarding tissue of the patient's body 160. To create axes-specific electric fields within body 160, the patch electrodes are placed on opposed surfaces of the body 160 (e.g., chest and back, left and right sides of the thorax, and neck and leg) and form generally orthogonal x, y, and z axes. A reference electrode/patch (not shown) is typically placed near the stomach and provides a reference value and acts as the origin of the first coordinate system 162 for the navigation system. Sinusoidal currents may be driven through each pair of patch electrodes 134 by electric signal generator 146, and voltage measurements for one or more position sensors 150 (e.g., electrodes 152, 154 or the tip electrode 170 located near the distal end 126 of catheter shaft 122) associated with the catheter 120 are obtained. The measured voltages are a function of the distance of the position sensors from the patch electrodes. The measured voltages are compared to the potential at the reference electrode and a position of the position sensors within the coordinate system 162 of the navigation system is determined.

The display 142 is provided to convey information to a physician to assist in diagnosis and treatment. The display 142 may comprise one or more conventional computer monitors or other display devices. The display 142 may present a graphical user interface (GUI) to the physician. The GUI may include a variety of information including, for example, an image of the geometry of the tissue/heart 180, electrophysiology data associated with the heart 180, graphs illustrating voltage levels over time for various sensors 150, and images of the catheter 120 and other medical devices and related information indicative of the position of the catheter 120 and other devices relative to the patient's body 160.

The ECU 144 provides a means for controlling the operation of various components of the system 100, including the catheter 120, an electric signal generator 146, and a switch 148 of the electric-field-based positioning system 136, and magnetic generator 130 of the magnetic-field-based positioning system 138. For example, the ECU 144 may be configured through appropriate software to provide control signals to switch 148 and thereby sequentially couple pairs of patch electrodes 134 to the signal generator 146. Excitation of each pair of electrodes 134 generates an electromagnetic field within the body 160 and within an area of interest such as the heart. The ECU 144 may also provide a means for determining the geometry of the tissue 180, electrophysiology characteristics of the tissue 180, and the position and orientation of the catheter 120 relative to tissue 180 and the body 160. The ECU 144 also provides a means for generating display signals used to control the display 142. The depicted ECU 144 represents any processing arrangement such as, for example, single device processors, multiple device processors (e.g., co-processors, master/slave processors, etc.), distributed processing across multiple components/systems, system on chip (SOC) devices, or the like.

FIGS. 2A-B illustrate diagrammatic view of a patient's heart 180 with a sensor enabled catheter 120 inserted therein, according to some embodiments. In the embodiment shown in FIG. 2A, the shaft 122 of the catheter 120 extends into a left ventricle of the patient's heart 180, however, it is contemplated that the shaft 122 of the catheter 120 may be positioned within or around other chambers or vessels of the patient's heart 180.

The sensor(s) 150 of the catheter 120 may include an electrode 170 on its distal tip configured to collect location data and/or tissue data. The sensor(s) 150 of the catheter 120 may include a pair of catheter electrodes 152, 154 configured to collect location data and/or tissue data, i.e., the electrodes 152, 154 may be configured to detect one or more characteristics of an impedance field/electrical field applied by a surface patch electrode 134. The sensor(s) 150 of the catheter 120 may include a magnetic sensor 156 configured to detect one or more characteristics of a magnetic field(s) applied by a magnetic field generator 130. The magnetic sensor 156 may include a coil configured to generate an electrical signal in response to the presence of a magnetic field.

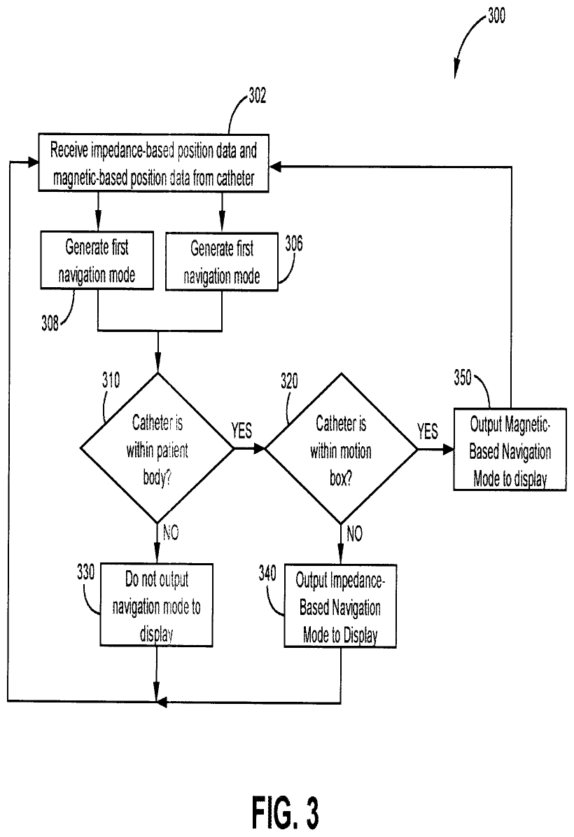

FIG. 3 illustrates a flow chart of a method 300 of collecting and presenting cardiac medical information to a physician using a catheter, according to some embodiments. At step 302, a first signal and a second signal is received from one or more sensors located on the catheter. The first signal may include impedance-based position and/or orientation data, and the second signal may include magnetic position and/or orientation data. In some embodiments, the first signal is received from one or more electrodes located at a distal end of the catheter and the second signal is received from a magnetic sensor located at the distal end of the catheter. The catheter electrode used in step 302 may include one or more of the catheter electrodes 152, 154, 170 illustrated in FIGS. 1-2B. In some embodiments, the catheter electrodes 152, 154, 170 may generate an electrical signal in response to movement through, or placement in, an electric field (or impedance field) generated by one or more surface patch electrodes 134. That is, the first signal may be related to signals received with respect to an impedance-based localization system. In other embodiments, the first signal received from the one or more catheter electrodes is unrelated to impedance-based localization measurements. For example, in some embodiments the first signal received from the catheter electrode may include a voltage, a current, an impedance, or a bipolar electrical complex impedance (BECI). In some embodiments, the first signal is generated in response to a signal driven between pairs of electrodes (e.g., catheter electrodes 152, 154, or between catheter electrode 152 and a surface patch electrode 134). For example, a bipolar electrode complex impedance (BECI) value is measured by driving an alternating current signal between an electrode pair, wherein at least one of the electrodes in the electrode pair is located on the catheter. As described in more detail below, a BECI value may be utilized to determine whether the catheter is located within the body.

The magnetic sensor used in the step 302 may include the magnetic sensor 156 illustrated in FIG. 2B. The magnetic sensor 156 may generate an electrical signal in response to movement through, or placement in, a magnetic field generated by the magnetic field generator 130. The second signal received from the magnetic sensor may include a voltage, a current, or other electrical signal. In some embodiments, the second signal received from the magnetic sensor may correlate to a position and/or orientation of the sensor within the magnetic motion box 132. The position and/or orientation of the magnetic sensor may be referred to as “magnetic-based” positioning/navigation, as the second signal is generated from the sensor's movement through magnetic field.

At step 306, a first navigation mode is generated. The first navigation mode (or “impedance-based navigation mode”) may be generated by receiving first electrical signals from the electrode and analyzing the first electrical signals with an ECU or processor. The first electrical signals may be converted into an impedance-based position point in a first coordinate system. The first navigation mode may track the position of one or more catheter electrodes (e.g., the electrodes 152, 154) over a plurality of time intervals and store the data within the ECU. In some embodiments, the first navigation mode may measure the output of one or more patch electrodes (e.g., patch electrodes 134).

At step 308, a second navigation mode is generated. The second navigation mode (or “magnetic-based navigation mode”) may be generated by receiving second electrical signals from the magnetic sensor and analyzing the second electrical signals with an ECU or processor. The second electrical signals may be converted into a magnetic-based position point in a second coordinate system. The second navigation mode may track the position of one or more magnetic sensors (e.g., the magnetic sensor 156) over a plurality of time intervals and store the data within the ECU. In some embodiments, the second navigation mode may measure the output of a magnetic field generator (e.g., magnetic field generator 130).

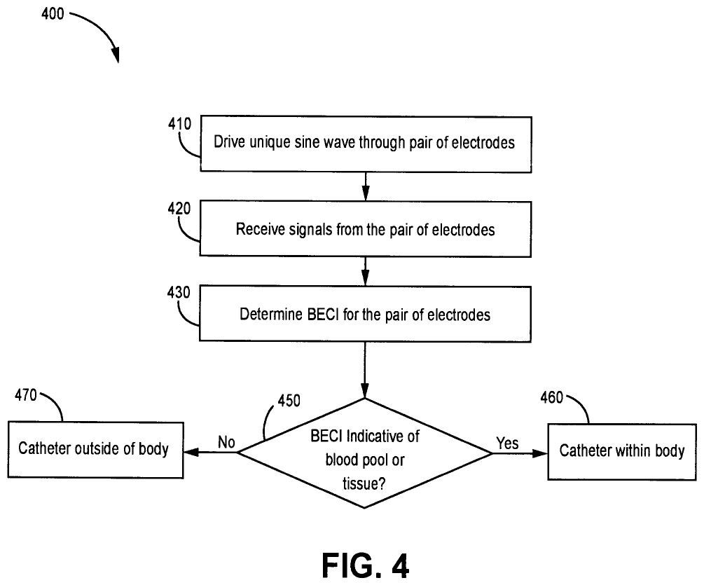

At step 310, a determination is made whether the catheter is located within a patient's body. In general, the term located within the patient's body refers to insertion of the catheter into the patient (i.e., through the surface of the skin) at any point on the patient (e.g., leg, chest, arms). In some embodiments, determining whether the catheter is within a patient body may include analyzing the received first and/or second signal(s) from the catheter electrode and/or magnetic sensor as described in the step 302. FIG. 4 provides an example of one method in which the first signals are utilized to determine whether the catheter is located within the patient's body. In some embodiments, only the first signal (e.g., signals received from the one or more catheter electrodes) are utilized in the determination of whether the catheter is located within the body. In some embodiments, the first signal is generated in response to the impedance-based localization system, in which the electrodes are utilized to sense the impedance field generated by a plurality of surface patch electrodes. In some embodiments, the presence (or absence) of a detected impedance field is utilized to determine whether the catheter is located within the body, wherein detected presence of the impedance field indicates the catheter is located within the body. In other embodiments, the actual location information ascertained from the first signal as part of an impedance-based localization system is utilized to determine whether the catheter is located within the body. In other embodiments, rather than utilize the impedance field generated by an impedance-based localization system, the first signal is generated in response to a signal generated between an electrode pair (at least one of which is located on the catheter) by driving a signal between the respective electrode pair. For example, an alternating current (AC) signal may be provided between the respective pair of electrodes and the resulting bipolar electrode complex impedance (BECI) value is calculated based on the measured response, wherein the BECI value represents the first signal. In some embodiments, the BECI value is utilized to determine whether the catheter is located within the body, wherein measured BECI value is representative of whether the one or more catheter electrodes are in contact with the blood pool (i.e., located within the body) or air and therefore NOT located within the body.

If a determination is made that the catheter is not within the patient's body at step 310, then at step 330 the display is prevented from outputting a navigation mode. In other words, if the catheter is not within a patient's body, the display (e.g., the display 142 of FIG. 3) will not show the catheter position and orientation. In some embodiments, the impedance-based localization system and/or the magnetic based localization system may be receiving inputs from the catheter and may be generating outputs indicating the location of the catheter, but the ECU prevents these outputs from being displayed to the display. In other embodiments, having determined that the catheter is not located within the patient's body at step 310, neither of the respective localization systems (e.g., impedance-based localization, magnetic-based localization) is made active. In this way, the display does not inadvertently indicate to the user that the catheter is positioned within the body. In some embodiments, steps 302 and 310 are repeated until it is determined that the catheter is located within the patient's body. In some embodiments, the loop may be set on a time-limit, i.e., steps 302, 310 may be repeated every t seconds, where t is an adjustable time value between 0.1 and 10 seconds.

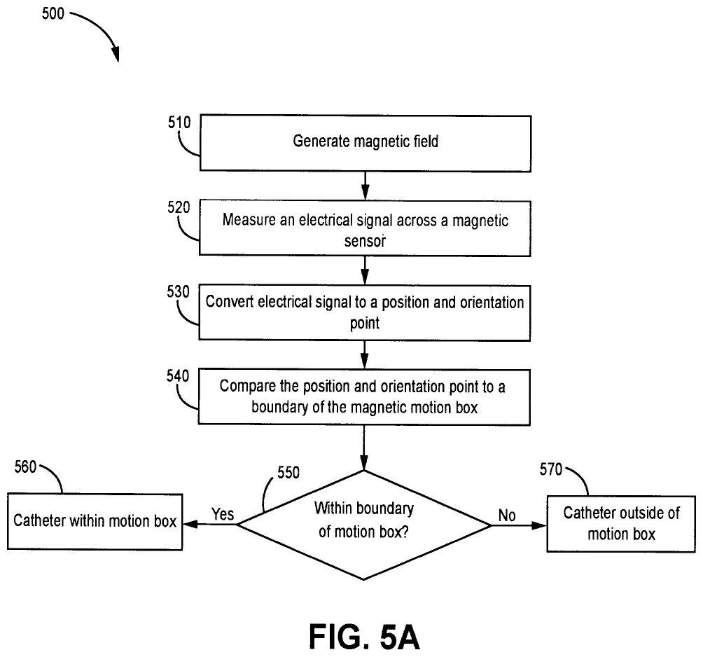

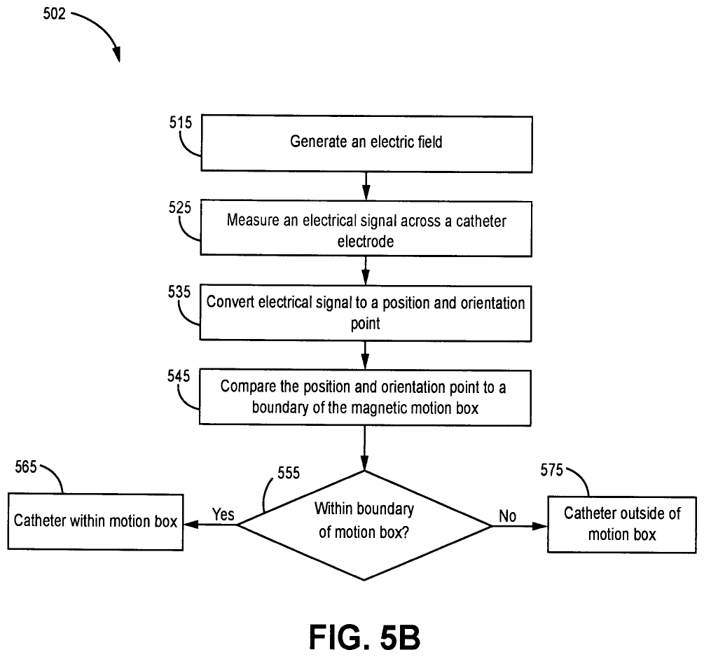

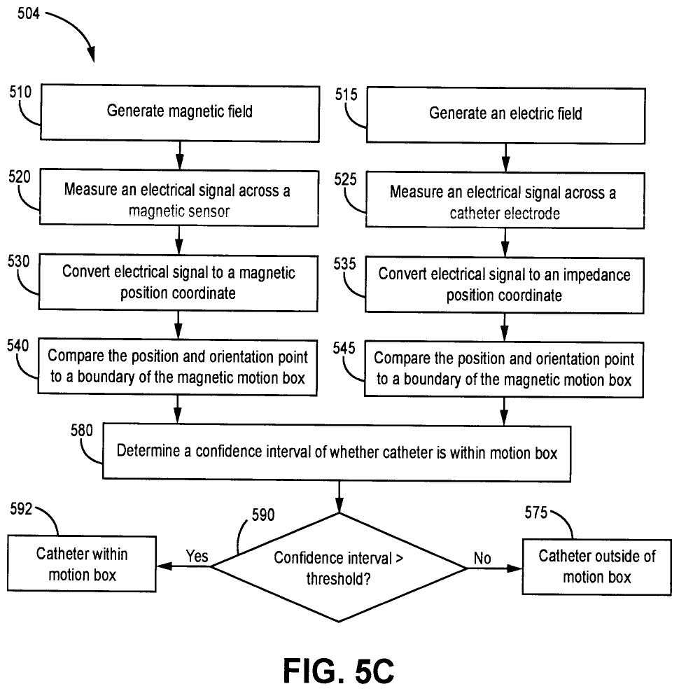

If a determination is made that the catheter is within the patient's body at step 310, then at step 320 a determination is made whether the catheter is within the magnetic motion box (e.g., the magnetic motion box 132 illustrated in FIG. 1). In some embodiments, determining whether the catheter is within a magnetic motion box may include analyzing the received signal(s) from the catheter electrode and/or magnetic sensor as described in the step 302. According to some embodiments, a method 500, 502, 504 (illustrated in FIGS. 5A-C) may be used to determine whether the catheter is within a magnetic motion box. In contrast with the determination at 310 to determine whether the catheter is within the patient's body which may be based on, for example, detecting whether the catheter electrodes are located within the blood pool, the determination of whether the catheter is located within the magnetic motion box depends on the determined location of the catheter. In this way, impedance-based localization based on signals received from the catheter electrode and/or magnetic-based localization based on signals received from the magnetic sensor may be utilized to determine whether the catheter is located within the magnetic motion box. In some embodiments, impedance-based localization is utilized to determine whether the catheter is located within the magnetic motion box. In some embodiments, magnetic-based localization is utilized to determine whether the catheter is located within the magnetic motion box. In some embodiments, a combination of impedance-based localization and magnetic-based localization is utilized to determine whether the catheter is located within the magnetic motion box (e.g., both must indicate that the catheter is located within the magnetic motion box).

If a determination is made that the catheter is within the patient's body (step 310) and not within the magnetic motion box (step 320), then at step 340 an impedance-based navigation mode is utilized to display the position of the catheter to the display. At step 340, the impedance-based data (i.e., the data from the first signal) of the catheter's position and orientation may be output to the display. For instance, the first signal may be received from the catheter electrode and impedance-based position and/or orientation data may be calculated from the first signal. The impedance-based position and/or orientation data may then be output to the display to provide a visual representation of the catheter's position and/or orientation within the patient's body. In some embodiments, a magnetic-based positioning system may be receiving second signals from the magnetic sensor and may be generating magnetic-based position and/or orientation information, but this information will not be output to the display so long as the system is operating in the impedance-based navigation mode.

Following the determination to output the impedance-based navigation mode to the display at step 340, the process continues to monitor at steps 302 and 310 whether the catheter is located within the patient's body. That is, the process may continue at steps 302, 310 to receive first and/or second signals to determine whether the catheter is located within the patient's body as described above.

If a determination is made that the catheter is within the patient's body (step 310) and within the magnetic motion box (step 320), then at step 350 a magnetic-based navigation mode is utilized to display the position of the catheter to the display. At step 350, the magnetic-based data (i.e., the data from the second signal) of the catheter's position and orientation may be output to the display. For instance, the second signal may be received from the magnetic sensor and magnetic-based position and/or orientation data may be calculated from the second signal. The magnetic-based position and/or orientation data may then be output to the display to provide a visual representation of the catheter's position and/or orientation within the patient's body. In some embodiments, the impedance-based positioning system may be receiving first signals from the one or more catheter electrodes and may be generating impedance-based position and/or orientation information, but this information will not be output to the display so long as the system is operating in the magnetic-based navigation mode. In some embodiments, at step 350 both magnetic-based positioning and impedance-based positioning may be utilized while operating within the magnetic motion box. For example, position and/or orientation may be output as an aggregate or combination of the magnetic-based positioning and impedance-based positioning.

Having determined that the catheter is located within the magnetic motion box and at step 350 displaying the location of the catheter using the magnetic-based navigation mode, the process continues to receive impedance-based position data and magnetic-based position data from catheter at step 302. The first and second navigation mode will continue to be generated (steps 306, 308), and determinations will continue to be made as to whether the catheter is within the patient's body (step 310) and whether the catheter is within the magnetic motion box (step 320). Thus, the method 300 may include a feedback loop after steps 330, 340, and 350 to continuously update step 310 and/or step 320, and generate an output accordingly. For instance, if the physician moves the catheter out of the magnetic motion box, the visualization/navigation system may automatically revert back to the impedance-based navigation mode. The method 300 may include a transition between the impedance-based navigation mode and the magnetic-based navigation mode when the catheter moves across a boundary of the magnetic motion box. Accordingly, the method 300 may allow the physician to transition between a first navigation mode and a second navigation mode within a single study (i.e., without resetting the navigation system).

In one example, as the physician moves the catheter from the incision site (e.g., the femoral artery or radial artery) to the heart, the catheter may first be determined to be within the patient body (step 310) and outside of the magnetic motion box (320), and the method 300 may proceed to step 340. At some point, the catheter may cross into the magnetic motion box, and thus, the catheter may be within the patient body (step 310) and within the magnetic motion box (320), and the method may proceed to step 350. Due to the automatic feedback loop(s) within the method 300, operation in the impedance-based navigation mode and/or magnetic-based navigation mode may be made automatically requiring feedback from the physician and/or notification to the physician. Or in other words, the physician will not need to stop the procedure to switch from an impedance-based navigation mode to a magnetic-based navigation mode. In some embodiments, the determination at steps 310 and 320 may prompt the physician to change navigation modes. For instance, if the catheter is moved from within the patient body and outside of the magnetic motion box (impedance-based navigation mode) to within the patient body and within the magnetic motion box, the display may include a prompt (e.g., an instruction or question) to direct the physician to select the magnetic-based navigation mode. Thus, the transition from one navigation mode to another may be subject to physician input, according to some embodiments.

In some embodiments, determining that the catheter is within the magnetic motion box (at step 320) may also determine that the catheter is within the patient body (step 310). For instance, if the magnetic motion box is entirely contained within the patient's body, i.e., only surrounding the patient's heart and/or not extending past the patient's chest cavity, a determination that the catheter is within the magnetic motion box will necessarily correlate to the catheter being within the patient's body.

FIG. 4 illustrates a method 400 of determining whether a catheter is located within a patient's body, according to some embodiments. In some embodiments, the step 310 of method 300 (see FIG. 3) may utilize the method 400. At step 410, an electrical signal is provided as an input to one or more of the catheter electrodes. In some embodiments, the electrical signal is provided to a single catheter electrode (unipolar, with a return path provided via a surface patch electrode, for example). In other embodiments, the electrical signal is provided between a pair of catheter electrodes (bipolar). For example, the pair of electrodes may include the catheter electrodes 152, 154 illustrated in FIG. 2B. In some embodiments, the electrical signal is an alternating current signal. In some embodiments, the frequency of the AC signal is within a given range (e.g., 16-19 kHz) and may be unique to each pair of catheter electrodes. In some embodiments, a signal generator (not shown) controlled by the ECU 144 may be configured to drive the unique sine wave through the pair(s) of electrodes.

At step 420 a signal is measured in response to the electrical signal provided at step 410. For example, in some embodiments a voltage is measured between the electrode pairs, and the measured voltage is communicated to the ECU 144. At step 430, a bipolar electrical complex impedance (BECI) is calculated based on the response measured at step 420 with respect to each catheter electrode and/or electrode pair. The BECI data may be collected and preprocessed by an amplifier device and the BECI measurements may be arranged as cosine/sine pair for each electrode. The BECI measurement for an electrode is dependent on the conductivity of the medium the electrode is in contact with. For instance, contact with a blood pool or tissue generate a relatively lower BECI than contact with air due to the lower impedance path provided between the electrodes by the blood pool and/or tissue.

The method 400 may include step 450, determining whether BECI is indicative of blood pool and/or tissue contact. Step 450 may include comparing a received BECI value to a threshold value. For instance, if a received BECI value exceeds the threshold value, the BECI value may be indicative of electrode contact with a blood pool or patient tissue. In contracts, if a BECI value falls below the threshold value, the BECI value may be indicative of electrode contact with air or a catheter sheath. If the BECI value is indicative of electrode contact with a blood pool or tissue, step 460 may generate an output indicating the catheter is within the patient's body. If the BECI value not indicative of electrode contact with a blood pool or tissue, step 470 may generate an output indicating the catheter is outside of the patient's body. In some embodiments, the threshold value may be pre-characterized static constant, or may be a dynamically characterized value through a baseline step. In some embodiments, the baseline step is performed per study.

In some embodiments, the method 400 may include a set of rules to determine whether the catheter is out of a patient's body. For instance, if one electrode of an electrode pair has a measurement higher than a characterized threshold (e.g., 0.5e³ ohms), then both electrodes forming the BECI pair are out of body. If more than X % of electrodes are out of body, then the whole catheter may be determined to be out of body, wherein X is a selectable value between 10-90. If a distal pair of electrodes include a BECI measurement indicative of out of body, then the whole catheter may be determined to be out of body.

In some embodiments, step 310 of the method 300 may include determining a position of the catheter based on the received first signal(s), i.e., based on the impedance-based position and orientation data. If the position of the catheter is determined to be located within the patient body based on the impedance-based position and orientation data, the method 300 may proceed to step 320. In some embodiments, the physician may manually determine (i.e., via the ECU 144 or a user interface on the display 142) that the catheter is within the patient's body.

FIGS. 5A-C illustrate flow charts of a method of determining whether a catheter is within a magnetic motion box, according to some embodiments. As illustrated in FIG. 5A, method 500 includes step 510, generating a magnetic field. The magnetic field may be generated by the magnetic field generator 130 illustrated in FIG. 1. The magnetic field may be generated to form a magnetic motion box (e.g., the magnetic motion box 132).

The method 500 may include step 520, receiving an electrical signal generated by the magnetic sensor in response to the magnetic field generated at step 510. The magnetic sensor may include the magnetic sensor 156 illustrated in FIG. 2B. The magnetic sensor may include a coil configured to generate a current (or electrical signal) as it moves through a generated magnetic field. The ECU 144 may measure the electrical signal from the magnetic sensor. At step 530, the electrical signal from the magnetic sensor is utilized to generate a position and/or orientation estimate of the magnetic sensor within the magnetic field. For instance, based on the received electrical signal, the ECU 144 may be configured to calculate a magnetic-based position and orientation of the magnetic sensor. The location of the magnetic sensor may be known relative to the catheter, and therefore, the catheter position and orientation may be determined via the received electrical signal.

At step 540, the measured position and orientation of the magnetic sensor is compared to a boundary of the magnetic motion box. In some embodiments, the magnetic motion box may be defined as a cuboid that encloses the heart of the patient as a 3D space with coordinates Xₘᵢₙ to Xₘₐₓ, Yₘᵢₙ to Yₘₐₓ, and Zₘᵢₙ to Zₘₐₓ. In other embodiments, the magnetic motion box may be defined by other geometric shapes and/or coordinate systems. Step 540 may include comparing the measured position to the coordinates of the magnetic motion box.

At step 550, a determination is made regarding whether the measured position of the magnetic sensor is within the magnetic motion box. If the measured position is within the 3D coordinates of the magnetic motion box, the method 500 may proceed to step 560, outputting a determination that the catheter is within the magnetic motion box. If the measured position is outside of the 3D coordinates of the magnetic motion box, the method 500 may proceed to step 570, outputting a determination that the catheter is outside of the magnetic motion box.

As illustrated in FIG. 5B, at step 515 a voltage/impedance field is generated. The voltage/impedance field may be generated by the generating a voltage across pairs of surface patch electrodes placed on the body of the patient. At step 525, an electrical signal is measured at one or more catheter electrodes. The catheter electrode may include the catheter electrodes 152, 154 illustrated in FIG. 2B. The electric signal may include a voltage, a current, an impedance, or other electrical signal.

At step 535, the electrical signal is utilized to determine a position of the electrode within the patient's body. For instance, based on the received electrical signal, the ECU 144 may be configured to calculate an impedance-based position of the catheter electrode. The location of two or more catheter electrode combined with geometry/shape information associated with the catheter may be utilized to determine an orientation of the catheter as well as position. In some embodiments, the position and orientation of the catheter electrodes, combined with information regarding the location of the magnetic sensor relative to the catheter electrodes, may be utilized to determine an impedance based location of the magnetic sensor separate from the magnetic-based position of the magnetic sensor determined based on signals generated in response to a magnetic field.

At step 545, the measured position of the catheter electrodes and/or magnetic sensor are compared to a boundary of the magnetic motion box. In some embodiments, the magnetic motion box may be defined as a cuboid that encloses the heart of the patient as a 3D space with coordinates. At step 555, a determination is made whether the measured position of the catheter electrode and/or magnetic sensor is within the motion box. If the measured position is within the 3D coordinates of the magnetic motion box, then at step 565 an output is generated indicating that the catheter electrodes and/or magnetic sensor is located within the motion box. If the measured position of the catheter electrodes and/or magnetic sensor is determined to be outside of the 3D coordinates of the magnetic motion box, then at step 575, an output is generated indicating that the catheter is located outside of the motion box.

FIG. 5C illustrates a method 504 of determining whether a catheter is within a magnetic motion box, according to some embodiments. The method 504 includes elements from methods 500 and 502, namely, by using both magnetic-based positioning (steps 510, 520, 530, 540) and impedance-based positioning (steps 515, 525, 535, 545) to determine whether the catheter is within a magnetic motion box.

Method 504 may include step 580, determining a confidence interval using magnetic-based positioning of the catheter and impedance-based positioning of the catheter. For instance, using the magnetic-based positioning (steps 510, 520, 530, 540), the method may determine a location of the catheter and assign a magnetic-based confidence value to the position (i.e., the system is X % confident that the catheter is within the motion box based on the magnetic-based positioning). Using the impedance-based positioning (steps 515, 525, 535, 545), the method may determine a location of the catheter and assign an impedance-based confidence value to the position (i.e., the system is Y % confident that the catheter is within the motion box based on the impedance-based positioning). The method 504 may utilize a weighted combination of the magnetic-based confidence value and the impedance-based confidence value to determine the confidence interval (step 580).

Step 590 may compare the calculated confidence interval to a threshold to determine whether the catheter is within the motion box. If the calculated confidence interval is greater than the threshold, the method 504 may proceed to step 592 and determine the catheter is within the motion box. If the calculated confidence interval is less than the threshold, the method 504 may proceed to step 575 and determine that the catheter is outside of the motion box.

The above Detailed Description includes references to the accompanying drawings, which form a part of the Detailed Description. The Detailed Description should be read with reference to the drawings. The drawings show, by way of illustration, specific embodiments in which the present devices, systems, and methods can be practiced. These embodiments are also referred to herein as “examples.”

The Detailed Description is intended to be illustrative and not restrictive. For example, the above-described examples (or one or more features or components thereof) can be used in combination with each other. Other embodiments can be used, such as by one of ordinary skill in the art upon reviewing the Detailed Description and accompanying drawings. Also, various features or components have been or can be grouped together to streamline the disclosure. This should not be interpreted as intending that an unclaimed disclosed feature is essential to any claim. Rather, inventive subject matter can lie in less than all features of a disclosed embodiment. Thus, the following claims are hereby incorporated into the Detailed Description, with each example standing on its own as a separate embodiment:
In Example 1, a method to collect and present cardiac medical information to a physician using a medical device includes receiving impedance-based position data from an electrode. A first navigation mode for visualizing the medical device is generated using the impedance-based position data. Magnetic-based position data is received from a magnetic sensor. A second navigation mode for visualizing the medical device is generated using the magnetic-based position data. A determination is made regarding whether the medical device is within a magnetic motion box generated by a magnetic field generator. One or more of the magnetic-based position data and the impedance-based position data is used to determine whether the medical device is within the magnetic motion box. The method includes outputting the first navigation mode to a display when the medical device is outside the magnetic motion box and outputting the second navigation mode to the display when the medical device is inside of the magnetic motion box.
In Example 2, the method of Example 1 is optionally configured to include transitioning between the first navigation mode and the second navigation mode when the catheter moves across a boundary of the magnetic motion box.
In Example 3, the method of Examples 1 and/or 2 is optionally configured such that the first navigation mode includes receiving a first electrical signal measured by the electrode. The first electrical signal is analyzed with an ECU. The first electrical signal is converted to a first position point in an impedance coordinate system.
In Example 4, the method of any of Examples 1-3 is optionally configured such that the second navigation mode includes receiving a second electrical signal generated by the magnetic sensor in response to an external magnetic field. The second electrical signal is analyzed with an ECU. The second electrical signal is converted to a second position point in a magnetic coordinate system.
In Example 5, the method of any of Examples 1-4 is optionally configured such that determining whether the medical device is within the magnetic motion box includes comparing the second position point of the magnetic sensor to a boundary of the magnetic motion box.
In Example 6, the method of any of Examples 1-5 is optionally configured such that determining whether the medical device is within the magnetic motion box includes comparing the first position point of the electrode to the boundary of the magnetic motion box.
In Example 7, the method of any of Examples 1-6 is optionally configured such that determining whether the medical device is within the magnetic motion box includes comparing the second position point of the magnetic sensor to a boundary of the magnetic motion box and comparing the first position point of the electrode to the boundary of the magnetic motion box.
In Example 8, the method of any of Examples 1-7 is optionally configured to include determining whether the medical device is within a patient body. Neither the first navigation mode nor the second navigation mode is output to the display when the medical device is outside of the patient body.
In Example 9, the method of any of Examples 1-8 is optionally configured such that determining whether the medical device is within a patient body includes driving an electrical signal through a first electrode and receiving a bipolar complex impedance on a second electrode. The bipolar electrical complex impedance is compared to a threshold value.
In Example 10, the method of any of Examples 1-9 is optionally configured such that transitioning between the first navigation mode and the second navigation mode occurs within a single study and includes a model conversion. The first position point in the impedance-based coordinate system is converted into the magnetic-based coordinate system.
In Example 11, the method of any of Examples 1-10 is optionally configured such that transitioning from the first navigation mode to the second navigation mode within the single study occurs automatically when the magnetic sensor is detected in the magnetic motion box.
In Example 12, the method of any of Examples 1-11 is optionally configured such that the physician is prompted to transition from the first navigation mode to the second navigation mode within the single study when the magnetic sensor is detected in the magnetic motion box.
In Example 13, the method of any of Examples 1-12 is optionally configured such that the first navigation mode and the second navigation mode are continuously and simultaneously computing a magnetic sensor location and an electrode location regardless of whether the first navigation mode or the second navigation mode is being output to the display.
In Example 14, the method of any of Examples 1-13 is optionally configured such that the magnetic motion box is a cuboid defining a three dimensional (3D) coordinate system, the cuboid configured to enclose a heart of a patient.
In Example 15, a medical imaging and navigation system includes a medical device, a pair of surface patch electrodes, a magnetic field generating assembly, a display, and a processor. The medical device includes a device electrode and a magnetic sensor. The pair of surface patch electrodes are positionable on a patient. The pair of surface patch electrodes are configured to generate an electric field within a patient body. A first electrical signal is measured on the device electrode due to the generated electric field. The magnetic field generating assembly is positioned adjacent the patient. The magnetic field generating assembly generates a magnetic field within a motion box. A second electrical signal is measured on the magnetic sensor due to the generated magnetic field. The processor receives the first electrical signal from the device electrode and determines a first position of the device electrode based on the first electrical signal. The processor stores the first position in a first dataset. The processor receives the second electrical signal from magnetic sensor and determines a second position of the magnetic sensor based on the second electrical signal. The processor stores the second position in a second dataset. The processor utilizes the first dataset to construct an impedance-based map study and utilizes the second dataset to construct a magnetic-based map study. The processor selectively outputs either the impedance-based map study or the magnetic-based map study to the user display based upon a location of the medical device within the patient body. The processor is configured to transition from the impedance-based map study to the magnetic-based map study within a single study.
In Example 16, the method of Example 15 is optionally configured such that the processor is configured to output the impedance-based map study to the user display when the medical device is within the patient body and outside of the motion box.
In Example 17, the method of any of Examples 15-16 is optionally configured such that the processor is configured to output the magnetic-based map study to the user display when the medical device is within the patient body and inside of the motion box.
In Example 18, the method of any of Examples 15-17 is optionally configured such that the processor determines whether the medical device is within the patient body by measuring a bipolar electrical complex range and comparing the bipolar electrical complex range to a characterized threshold.
In Example 19, the method of any of Examples 15-18 is optionally configured such that the characterized threshold is a dynamically characterized value established through a baseline step.
In Example 20, the method of any of Examples 15-19 is optionally configured such that the user display includes a user interface configured to allow a physician to select between a first navigation mode and a second navigation mode within the single study.

Certain terms are used throughout this patent document to refer to features or components. Different people may refer to the same feature or component by different names. This patent document does not intend to distinguish between components or features that differ in name but not in function.

The scope of the present devices, systems, and methods should be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended; that is, a device, system, or method that includes features or components in addition to those listed after such a term in a claim are still deemed to fall within the scope of that claim. Moreover, in the following claims, the terms “first,” “second” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

What is claimed is:

1. A method to collect and present cardiac medical information to a physician using a medical device, the method comprising:
receiving impedance-based position data from an electrode;
generating a first navigation mode for visualization of the medical device using the impedance-based position data;
receiving magnetic-based position data from a magnetic sensor;
generating a second navigation mode for visualization of the medical device using the magnetic-based position data;
determining whether the medical device is within a magnetic motion box generated by a magnetic field generator using one or more of the magnetic-based position data and the impedance-based position data; and
outputting the first navigation mode to a display when the medical device is outside the magnetic motion box and the second navigation mode to the display when the medical device is inside of the magnetic motion box.

2. The method of claim 1, further comprising transitioning between the first navigation mode and the second navigation mode when the medical device moves across a boundary of the magnetic motion box.

3. The method of claim 1, wherein generating the first navigation mode includes:
receiving a first electrical signal measured by the electrode;
analyzing the first electrical signal with an Electronic Control Unit (ECU); and
converting the first electrical signal to a first position point in an impedance-based coordinate system.

4. The method of claim 3, wherein generating the second navigation mode includes:
receiving a second electrical signal generated by the magnetic sensor in response to an external magnetic field;
analyzing the second electrical signal with the ECU; and
converting the second electrical signal to a second position point in a magnetic-based coordinate system.

5. The method of claim 4, wherein determining whether the medical device is within the magnetic motion box includes:
comparing the second position point of the magnetic sensor to a boundary of the magnetic motion box.

6. The method of claim 4, wherein determining whether the medical device is within the magnetic motion box includes:
comparing the first position point of the electrode to a boundary of the magnetic motion box.

7. The method of claim 4, wherein determining whether the medical device is within the magnetic motion box includes:
comparing the first position point of the electrode to a boundary of the magnetic motion box, and
comparing the second position point of the magnetic sensor to the boundary of the magnetic motion box.

8. The method of claim 1, further comprising determining whether the medical device is within a patient body, wherein neither the first navigation mode nor the second navigation mode is output to the display when the medical device is outside of the patient body.

9. The method of claim 8, wherein determining whether the medical device is within the patient body includes:
driving an electrical signal through a first electrode;
measuring a voltage signal on a second electrode;
calculating a bipolar electrical complex impedance with the measured voltage signal; and
comparing the bipolar electrical complex impedance to a threshold value.

10. The method of claim 4, wherein transitioning between the first navigation mode and the second navigation mode occurs within a single study and includes a model conversion wherein the first position point in the impedance-based coordinate system is converted into the magnetic-based coordinate system.

11. The method of claim 10, wherein transitioning from the first navigation mode to the second navigation mode within the single study occurs automatically when the magnetic sensor is detected in the magnetic motion box.

12. The method of claim 10, wherein the physician is prompted to transition from the first navigation mode to the second navigation mode within the single study when the magnetic sensor is detected in the magnetic motion box.

13. The method of claim 1, wherein the first navigation mode and the second navigation mode are continuously and simultaneously computing a magnetic sensor location and an electrode location regardless of whether the first navigation mode or the second navigation mode is being output to the display.

14. The method of claim 1, wherein the magnetic motion box is a cuboid defining a three dimensional (3D) coordinate system, the cuboid configured to enclose a heart of a patient.

15. A medical imaging and navigation system comprising:
a medical device including a catheter electrode and a magnetic sensor;
a pair of surface patch electrodes positionable on a patient, the pair of surface patch electrodes configured to generate an electric field within a patient body, wherein a first electrical signal is measured on the device electrode due to the generated electric field;
a magnetic field generating assembly positioned adjacent the patient, the magnetic field generating assembly configured to generate a magnetic field within a magnetic motion box, wherein a second electrical signal is measured on the magnetic sensor due to the generated magnetic field;
a display;
a processor configured to receive the first electrical signal from the device electrode and determine a first position point of the device electrode based on the first electrical signal, the processor configured to store the first position point in a first dataset, wherein the processor is configured to receive the second electrical signal from magnetic sensor and determine a second position point of the magnetic sensor based on the second electrical signal, the processor configured to store the second position point in a second dataset,
wherein the processor utilizes the first dataset to construct an impedance-based map study and utilizes the second dataset to construct a magnetic-based map study,
wherein the processor selectively outputs either the impedance-based map study or the magnetic-based map study to the display based upon a location of the medical device within the patient body, wherein the processor is configured to transition from the impedance-based map study to the magnetic-based map study within a single study.

16. The medical imaging and navigation system of claim 15, wherein the processor is configured to output the impedance-based map study to the display when the medical device is within the patient body and outside of the magnetic motion box.

17. The medical imaging and navigation system of claim 16, wherein the processor is configured to output the magnetic-based map study to the display when the medical device is within the patient body and inside of the magnetic motion box.

18. The medical imaging and navigation system of claim 15, wherein the processor determines whether the medical device is within the patient body by measuring a bipolar electrical complex impedance and comparing the bipolar electrical complex impedance to a threshold value.

19. The medical imaging and navigation system of claim 18, wherein the threshold value is a dynamically characterized value established through a baseline step.

20. The medical imaging and navigation system of claim 15, wherein the display includes a user interface configured to allow a physician to select between a first navigation mode and a second navigation mode within the single study.
